(19) United States
(12) Patent Application Publication
(10) Pub. No.: US 2003/0236553 A1
(43) Pub. Date: Dec. 25, 2003

(54) Thermotropic polymer heterofilament

(75) Inventor: Robert B. Knudsen, Charlotte, NC (US)

Correspondence Address: ROBERT H. HAMMER III, P.C., 3121 SPRINGBANK LANE, SUITE I, CHARLOTTE, NC, 28226, US

(73) Assignee: Celanese Advanced Materials, Inc.

(21) Appl. No.: 10/178,115

(22) Filed: Jun. 24, 2002

(51) Int. Cl. A61B 17/04

(52) U.S. Cl. 606/228

(57) ABSTRACT

A sheath/core heterofilament has a core polymer and a sheath polymer. The core polymer and the sheath polymer are different. One of the polymers is a thermotropic polymer. The other polymer is preferably a thermoplastic polymer selected from the group consisting of polyamides, polyesters, polyolefins, fluorocarbons, and combinations thereof. The weight ratio of the core polymer to the sheath polymer ranges from 50:50 to 80:20. When the core polymer is the thermotropic polymer, the sheath polymer is adapted to contain microfibrils formed by the thermotropic polymer core. These heterofilaments may be used as sutures or as orthopedic (or surgical) cords.

### FIELD OF THE INVENTION

This invention is directed to a sheath/core heterofilament, particularly for use in medical and veterinary applications, such as sutures and orthopedic (or surgical) cords.

### BACKGROUND OF THE INVENTION

Wound healing is facilitated by the use of sutures and orthopedic (or surgical) cords. These devices are used to hold the wound together, so that the body's natural healing process can knit together the wound. The ideal suture material would be: sterile; non-electrolytic, non-capillary, non-allergenic, and non-carcinogenic; non-ferromagnetic; easy to handle, minimally reactive in tissue and not predisposed to bacterial growth; capable of holding tissue layers throughout the critical wound healing period securely when knotted without fraying or cutting; resistant to shrinking in tissues; and absorbed completely with minimal tissue reaction after serving its purpose. The ideal suture, however, does not exist, so the surgeon must select an existing suture as close to ideal as possible and maintain the following qualities: high uniform tensile strength, permitting use of finer sizes; high tensile strength retention in vitro, holding the wound securely throughout the critical healing period; consistent uniform diameter; sterile; pliable for ease of handling and knot security; freedom from substances or impurities for optimum tissue acceptance; and predicable performance. ETHICON Wound Closure Manual, Page 24.

Fibers made of thermotropic polymer would be excellent candidates for suture materials. Such fibers have excellent tensile strength and cut resistance, and great resistance to creep. These fibers, however, express microfibrils when bent or abraded. These microfibrils can then penetrate the surrounding soft tissue and cause sinus formation. Such a sinus could collect contaminants or foreign matter, and subsequently become infected. Accordingly, thermotropic polymer fibers are not preferred in some medical and veterinary applications.

Accordingly, there is a need for new suture materials and orthopedic (or surgical) cords that are closer to the ideal suture material.

### SUMMARY OF THE INVENTION

A sheath/core heterofilament has a core polymer and a sheath polymer. The core polymer and the sheath polymer are different. One of the polymers is a thermotropic polymer. The other polymer is preferably a thermoplastic polymer selected from the group consisting of polyamides, polyesters, polyolefins, fluorocarbons, and combinations thereof. The weight ratio of the core polymer to the sheath polymer ranges from 50:50 to 80:20. When the core polymer is the thermotropic polymer, the sheath polymer is adapted to contain microfibrils formed by the thermotropic polymer core. These heterofilaments may be used as sutures or as orthopedic (or surgical) cords.

### DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1 is a monofilament heterofil with a sheath/core cross-section.

FIG. 2 is a multifilament of heterofils.

FIG. 3 is a heterofil having a multifilament core.

### DETAILED DESCRIPTION OF THE INVENTION

A heterofilament (or heterofil or bicomponent fiber), as used in this application, is a manufactured fiber made from two different polymer components. One polymer component is a thermotropic polymer. Specifically, in this application, the heterofilament has a sheath/core construction. A preferred heterofilament has a weight ratio of core polymer to sheath polymer in the range of 50:50 to 80:20. The heterofilament may be a monofilament, a multifilament of heterofilaments, or a monofilament having a multifilament core. These various heterofilaments will be illustrated below.

A thermotropic polymer exhibits liquid crystal formation in melt form. In thermotropic polymers, there must be a balance between having the necessary degree of molecular perfection to preserve the liquid crystal formation and the amount of imperfection to permit melting at workable temperatures. These polymers give high-modulus, highly oriented extrusion products. One such material is commercially available, in resin form, under the tradename VECTRA® from Ticona LLC, Summit, N.J. Another such material is commercially available, in fiber form, under the tradename VECTRAN® from Celanese Advanced Materials, Inc., Charlotte, N.C. VECTRAN multifilament yarns with a 1500 denier have a diameter of about 0.38 mm, 900 denier—about 0.3 mm, and 650denier—about 0.25 mm. VECTRAN multifilament yarns are also available in 200 denier (40 filaments at 5 dpf (denier per filament)) and 100 denier (20 filaments at 5 dpf). Each filament has a diameter of about 23 microns.

The other polymer is preferably adapted to contain microfibrils that is preventing the microfibrils from the thermotropic polymer from penetrating the surrounding soft tissue and forming sinuses. Preferably, any thermoplastic polymer with good biocompatibility may be used in medical and veterinarial applications. Examples of such polymers may be selected from the group consisting of polyamides, polyesters, polyolefins, fluorocarbons, and combinations thereof. Polyolefins include, but are not limited to, polyethylene, polypropylene and polybutylene. Polyethylene and polypropylene are most preferred. Fluorocarbons include, but are not limited to, polytetrafluoroethylene (PTFE). Polyesters include, but are not limited to, polyethylene terephthalate (PET). Polyamides include, but are not limited to, nylon. By containing the microfibrils and thereby eliminating sinus formation, the physical (e.g., tensile and cut-resistant) properties of the thermotropic polymers can be fully utilized in the medical and veterinary applications.

A suture is a thread used to stitch parts of a body together. A suture may have a diameter ranging from 0.079 mm (0.0031 inches or U.S.P. 6-0 or Brown & Sharpe 40) to 0.92 mm (0.0363 inches or U.S.P. 6 or Brown & Sharpe 19).

An orthopedic (or surgical) cord is used, for example, to join bones together or a bone to a tissue, such as a ligament or muscle, e.g. to temporarily replace bone during healing. For example, during open-heart surgery, the sternum is cut so that the rib cage may be spread open. Upon completion, the sternum must be joined together and held fast, so that it can heal (knit together). A cord of the foregoing heterofilaments may used to fasten the sternum together. These types of applications are most often used where a tensile member is needed to facilitate healing. Another example would be in the repair of a fractured ankle. Historically, stainless steel wire was used in these types of applications, but in prolonged use, it fails by fatiguing and fractures into small pieces that are detrimental to healing. Other examples of the use of such cords are set forth in U.S. Pat. Nos. 5,456,722 and 5,540,703, incorporated herein by reference, and include the repair of a patella fracture and the fusion of spinal vertebrae.

The cord construction is not limited. The cord may include parallel, braided, or twisted constructions. Such a cord may have 4-36 strands per braid. The cord may have 0-40 picks per inch.

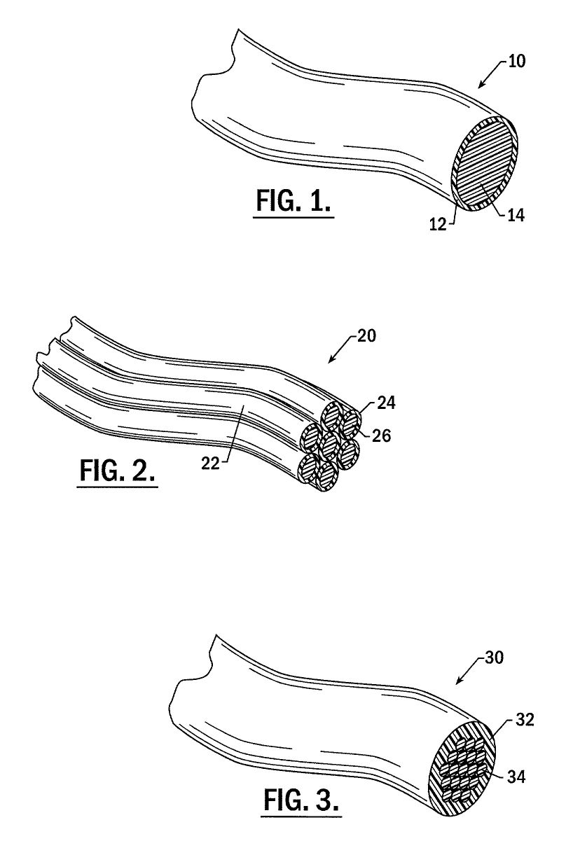

Referring to the drawings, there is shown in FIG. 1 a monofilament heterofilament 10. Heterofilament 10 has a sheath 12 and a core 14. Sheath 12 is a polymer that is adapted to contain microfibrils and may be selected from the group consisting of polyamides, polyesters, and polyolefins, and combinations thereof. Core 14 is made of a thermotropic polymer.

FIG. 2 illustrates a plurality of heterofilament 20. Each heterofilament 22 consists of a sheath 24 and a core 26. Sheath and core materials are as discussed above. The plurality of heterofilaments may be twisted or braided. The number of heterofilaments can be adjusted to the desired diameter.

FIG. 3 illustrates a heterofilament 30 having a core of multifilaments. Sheath 32 is made of the materials discussed above. Multifilament core 34 consists of a plurality of multifilaments made from a thermotropic polymer. Such a heterofilament may be made according to the method set forth in U.S. Pat. No. 5,601,775. The number of multifilaments in the core can be adjusted to the desired diameter.

The present invention may be embodied in other forms without departing from the spirit and the essential attributes thereof, and, accordingly, reference should be made to the appended claims, rather than to the foregoing specification, as indicated the scope of the invention.

In the claims:

1. A heterofilament comprises a core of a thermotropic polymer and a sheath of a polymer adapted to contain microfibrils formed by said core of thermotropic polymer.

2. The heterofilament of claim 1 wherein said polymer being selected from the group consisting of polyamides, polyesters, polyolefins, fluorocarbons, and combinations thereof.

3. The heterofilament of claim 1 wherein a weight ratio of core polymer to sheath polymer ranges from 50:50 to 80:20.

4. The heterofilament of claim 1 being a monofilament.

5. The heterofilament of claim 1 being a multifilament.

6. The heterofilament of claim 1 wherein said core comprises a multifilament yarn of said thermotropic polymer.

7. The heterofilament of claim 4 being a suture.

8. The heterofilament of claim 5 being a suture.

9. The heterofilament of claim 6 being a suture.

10. The heterofilament of claim 4 being a part of an orthopedic cord.

11. The heterofilament of claim 5 being a part of an orthopedic cord.

12. The heterofilament of claim 6 being a part of an orthopedic cord.

13. A heterofilament comprises a core polymer and a sheath polymer, said core polymer and said sheath polymer being different, and one said polymer being a thermotropic polymer.

14. The heterofilament of claim 13 wherein said other polymer being selected from the group consisting of polyamides, polyesters, polyolefins, fluorocarbons, and combinations thereof.

15. The heterofilament of claim 13 wherein a weight ratio of core polymer to sheath polymer ranges from 50:50 to 80:20.

16. The heterofilament of claim 13 being a monofilament.

17. The heterofilament of claim 13 being a multifilament.

18. The heterofilament of claim 13 wherein said core comprises a multifilament yarn.

19. The heterofilament of claim 16 being a suture.

20. The heterofilament of claim 17 being a suture.

21. The heterofilament of claim 18 being a suture.

22. The heterofilament of claim 16 being a part of an orthopedic cord.

23. The heterofilament of claim 17 being a part of an orthopedic cord.

24. The heterofilament of claim 18 being a part of an orthopedic cord.
